(19) United States
(12) Patent Application Publication
(10) Pub. No.: US 2013/0023881 A1
(43) Pub. Date: Jan. 24, 2013

(54) Surgical Kit and Method for Bone Repair

(71) Applicants: Guy S. Cook, Bozeman, MT (US); Wayne K. Gersoff, Parker, CO (US); Craig S. Smith, Bozeman, MT (US)

(72) Inventors: Guy S. Cook, Bozeman, MT (US); Wayne K. Gersoff, Parker, CO (US); Craig S. Smith, Bozeman, MT (US)

(21) Appl. No.: 13/185,920

(22) Filed: Jul. 19, 2011

(51) Int. Cl. A61B 17/16; A61B 17/17

(52) U.S. Cl. 606/80; 606/96

(57) ABSTRACT

A kit of surgical instruments for bone repair includes a set of sizer instruments, a set of drill bits for bone boring and a set of guide instruments. The sizer instruments are defined by bodies of varying diameters. The drill bits have varying diameters that correspond to the sizer instruments. The guide instruments are defined by thin-walled cylindrical bodies of varying diameters that correspond to the diameters of the sizer instruments and drill bits. Each cylindrical body includes an open proximal end and an open distal end and has an inner cylindrical surface sized to provide for translating movement of a corresponding sizer instrument and drill bit instrument therethrough. In one embodiment, the sizer instruments, the drill bits and the guide instruments of the kit each have a feature that is color coded to provide a visual indication of corresponding sized instruments of the kit. In another embodiment, each given drill bit has a plurality of distal surfaces that are oriented in respective planes that are substantially transverse to the central axis of the given drill bit such that given drill bit cuts a hole into bone wherein the hole has a substantially flat bottom surface. In yet another embodiment, each given guide instrument includes at least one window in the cylindrical body that provides for visualization into the interior space of the given guide instrument during use. Methods of bone repair using the kit is also disclosed and claimed.

### BACKGROUND OF THE INVENTION

1. Field of the Invention

The present invention relates to bone repair. More specifically, the present invention relates to bone repair for orthopedic applications, such as repairing defects in the subchondral region of articulating joints, although it is not limited thereto.

2. State of the Art

Bone grafting is a surgical procedure that promotes bone healing after facture, bone loss, infection, tumor, or other pathologic conditions. There are four broad clinical situations in which bone grafting is performed. First, it is used to stimulate healing of fractures—either fresh fractures or fractures that have failed to heal after an initial treatment attempt. Second, it is used to stimulate healing between two bones across a diseased joint. This situation is called “arthrodesis” or “fusion”. Third, it is used to regenerate bone and/or cartilage which is lost or missing or defective as a result of trauma, infection, or disease (such as subchondral defects). Fourth, it is used to improve the bone healing response and regeneration of bone tissue around surgically implanted devices, such as artificial joints replacements (e.g., total hip replacement or total knee replacement) or plates and screws used to hold bone alignment.

Bone grafts may utilize autograft bone (bone harvested from the patient's own body, often from the iliac crest), allograft bone (cadaveric bone usually obtained from a bone bank), or synthetic bone (often made of hydroxyapatite or other naturally-occurring and biocompatible substances) with mechanical properties similar to bone.

As appreciated by those skilled in the art, there are many advantages for using autograft bone in bone defect repair. For example, autograft bone is typically viscoelastic, osteoconductive, osteoinductive, and osteogenic (i.e., contains cells in its matrix that promote bone formation). In addition, autograft bone avoids histocompatibility and infectious disease issues. Autograft bone, however, is limited in supply, is generally painful to the patient upon harvesting, and may lead to significant donor site morbidity (i.e., may require additional surgical incisions in the patient, may lead to surgical complications, blood loss and may cause additional patient discomfort, and may ultimately increase patient recovery time).

Allograft bone is advantageous from the standpoint of being available in larger quantities compared to autograft bone. However, allograft bone may present disadvantages relating to histocompatibility issues (e.g., rejection by recipient immune system), the potential harboring of infectious agents, and may also include bone with poor malleable or mechanical characteristics (e.g., elasticity, compressibility, resiliency, and the like) due to high calcium and mineral content.

Synthetic bone can suffer from many of the same disadvantages as outlined above with regard to allograft bone. Synthetic bone products are generally formulated as putty and gel-type fillers, designed to be inserted into open space(s) between bone defects (i.e., defect or void fillers). Traditionally, synthetic bone products are made from allogeneic bone chips, granules, or bone powder, or other synthetic materials with or without carrier compositions.

Additionally, xenogeneic bone graft products are commercially available that are made from bovine bone. Disadvantages are similar to those presented with allograft bone, including a potential immune reaction to the xenogeneic bone and infectious agents, including prions.

The assignee of the present invention currently sells a bone graft product under the trademark OsteoSponge®. The OsteoSponge® bone graft product consists of 100% human cancellous bone that has been demineralized to provide a value-added benefit over other allograft bone material. The demineralization assists with two functions: it makes the product compressible so it is ideal for surgeons to obtain a press-fit, precise placement of the graft, and it exposes the native growth factors and bone morphogenic proteins that are essential for new bone formation. It also maintains the natural interconnected porosity of cancellous bone providing an ideal scaffold for cellular infiltration and bone formation. The OsteoSponge® bone graft product has the following advantages:
It is 100% bone; there is no carrier material.
It is osteoconductive in that it provides a natural interconnected, porous, cancellous scaffold with increased surface area for better cellular ingrowth, exposing bone-growth-inducing proteins to the healing environment.
It is osteoinductive in that it provides a host of signaling molecules that are involved in triggering the formation of new bone.
It is elastic and malleable.
It may be shaped and compressed to fit in and around a variety of voids or devices; when compressed and inserted into a bone void, the graft will expand to fill the contours of the void, thus minimizing the space of the void-graft interface.
It is provided sterile in its final packaging.
It has a convenient shelf life in that it is provided with a 5 year shelf life when stored at room temperature.
It can be used as a delivery vehicle for agents, such as bone marrow aspirate, platelet-rich plasma or other cellular concentrates.

During most arthroscopic bone repair procedures, the defective bone is removed by motorized drill or bur (and possibly curette tools for large fragments). A bone graft product is held by a grasper and introduced into the operative site via manipulation of the grasper. In these procedures, it can be difficult to properly identify the size of the defect, to remove all of the defective bone, and effectively size the bone graft for the defect and accurately place the bone graft within the defect.

### SUMMARY OF THE INVENTION

The problems of the prior art are solved by a kit of surgical instruments for bone repair that includes a set of sizer instruments, a set of drill bits for bone boring, and a set of guide instruments. The sizer instruments of the kit are defined by bodies of varying diameters. The drill bits of the kit have varying diameters that correspond to the sizer instruments of the kit. The guide instruments of the kit are defined by thin-walled cylindrical bodies of varying diameters that correspond to the diameters of the sizer instruments and drill bits of the kit. Each cylindrical body includes an open proximal end and an open distal end and has an inner cylindrical surface sized to provide for translating movement of a corresponding sizer instrument and drill bit instrument therethrough. The instruments of the kit provide for efficient and effective bone repair, particularly for repair of defective bone in the subchondral region of articulating joints, although the invention is not limited thereto.

In one embodiment, the sizer instruments, the drill bits and the guide instruments of the kit each have a feature that is color coded to provide a visual indication of corresponding sized instruments of the kit. Preferably, a respective color is painted or otherwise inscribed on the feature of corresponding sized sizer instruments, drill bits, and guide instruments of the kit to provide a visual indication of corresponding sized instruments of the kit. The feature is preferably defined about the circumference of an outer surface of a respective instrument of the kit, but may be applied to the entire instrument or particular surfaces of the instrument. In the preferred embodiment, the feature comprises a band or groove that extends about the circumference of the outer surface of the respective instrument of the kit. Alphanumeric characters that convey size can also be marked on the corresponding sized sizer instruments, drill bits, and guide instruments of the kit to provide a visual indication of corresponding sized instruments of the kit.

In the preferred embodiment, the sizer instruments and the drill bits of the kit each have a central lumen that extends along their respective entire length. The central lumen of the respective instrument allows for passage of the respective instrument over an elongate fixation member that has a distal end that is adapted to be fixed into bone. The kit can include one or more such fixation members.

In another embodiment, each given drill bit of the kit has a plurality of distal surfaces that are oriented in respective planes that are substantially transverse to the central axis of the given drill bit such that given drill bit cuts a hole into bone wherein the hole has a substantially flat bottom surface.

In yet another embodiment, each given guide instrument of the kit includes at least one window in the cylindrical body of the given guide instrument in order to provide for visualization into the interior space of the given guide instrument during use. The at least one window is preferably narrow in shape and extends parallel to the central axis of the cylindrical body of the given guide instrument. In the preferred embodiment, the at least one window comprises a singular or plurality of windows that are narrow in shape and extend parallel to the central axis of the cylindrical body of the given guide instrument. Some of the windows can be spaced apart from one another in a direction parallel to the central axis of the cylindrical body, and other windows can be spaced apart from one another about the circumference of the cylindrical body of the given guide instrument. The edges of the plurality of windows can be chamfered and/or deburred.

The surgical kit can be used in a surgical method for the repair of a bone defect. More specifically, the sizer instruments of the kit are used to identify a proper sizer instrument that covers the bone defect. A proper guide instrument and a proper drill bit of the kit are selected that match the proper sizer instrument. The proper guide instrument is used to guide movement of the proper drill bit during coring of the bone defect in order to drill a hole in the bone defect. A bone graft is selected that is sized for the hole drilled in the bone defect. The proper guide instrument is used to guide introduction of the bone graft into the hole drilled in the bone defect.

In one embodiment, the bone graft is pushed with the distal end of the proper sizer through the proper guide instrument in order to introduce the bone graft into the hole drilled in the bone defect.

In another embodiment, a fixation member is inserted through a central lumen defined by the proper sizer instrument. The distal end of the fixation member is secured in a central region of the bone defect. After the distal end of the fixation member is secured in the central region of the bone defect, the proper drill bit can be moved over the fixation member during coring of the bone defect in order to drill a hole in the bone defect. After such drilling is complete and the drill bit removed from fixation member, a bone graft may be introduced over the fixation member and the proper sizer can be used to push the bone graft with the distal end of the proper sizer through the proper guide instrument for introducing the bone graft into the hole drilled in the bone defect.

In one embodiment, the surgical method employs a bone graft defined by a body of bone material with a side surface sized for insertion into the hole drilled into the bone defect. The body also includes a central thru-hole that allows for passage of a fixation member therethrough for guided placement of the block into the drilled hole. The bone material of the bone graft is preferably realized from demineralized cancellous bone matrix. Alternatively, the material of the bone graft can be selected from the group consisting of: autograft bone, allograft bone, a synthetic bone product, and a xenogeneic bone product.

### BRIEF DESCRIPTION OF THE DRAWINGS

FIG. 1A is an isometric view (particularly of the distal end) of a sizer instrument of a surgical kit and bone graft methodology according to the present invention.

FIG. 1B is an isometric view (particularly of the proximal end) of the sizer instrument of FIG. 1A.

FIG. 1C is an enlarged view of the proximal end of the sizer instrument of FIG. 1A.

FIG. 1D is a plan view of the distal end of the sizer instrument of FIG

FIG. 2 is an isometric view of a fixation member of a surgical kit and bone graft methodology according to the present invention.

FIG. 3A is an isometric view (particularly of the distal end) of a guide instrument of a surgical kit and bone graft methodology according to the present invention.

FIG. 3B is a side view of the guide instrument of FIG. 3A.

FIG. 4A is an isometric view (particularly of the distal end) of a drill bit instrument of a surgical kit and bone graft methodology according to the present invention.

FIG. 4B is a side view of the drill bit instrument of FIG. 4A.

FIG. 4C is an enlarged view of the distal end of the drill bit instrument of FIG. 4A.

FIG. 4D is an enlarged view of the proximal end of the drill bit instrument of FIG. 4A.

FIG. 5 is a schematic view of a surgical tray for use as part of the surgical kit of the present invention.

### DETAILED DESCRIPTION OF THE PREFERRED EMBODIMENTS

The term “demineralized cancellous bone matrix” or “matrix” as used herein refers to a porous extracellular matrix of cancellous bone remaining after demineralization. Such a matrix provides scaffolding conducive for cell attachment and tissue regeneration and can be used to promote bone healing, arthrodesis, new bone formation, and repair of pathologic non-union. An example of a demineralized cancellous bone matrix is the OsteoSponge® bone graft product sold commercially by the assignee of the present invention.

Demineralization removes the inorganic mineral component of a piece of cancellous bone, for example by subjecting the bone to an acid solution as described in U.S. Pat. Publ. No. 2008/0305145, commonly assigned to assignee of the present application and incorporated by reference in its entirety. After acid treatment, the matrix can be subjected to a base solution to raise the pH of the matrix. The matrix can be stored under aseptic conditions until it is used and/or can be sterilized before use. In the preferred embodiment, the matrix has a residual mineral content less than two percent. The matrix can be compressible. Preferably, the matrix is compressible to less than sixty percent of its size at rest using compression forces between 10 and 100 grams-force per square cm. In one embodiment, the matrix has special properties that may include one or more of:
i) The matrix is biocompatible and can provide a natural scaffold for cellular ingrowth and proliferation; the matrix also retains growth factors that signal infiltrating host cells to initiate the formation of new blood vessels and to differentiate into osteoclasts and osteoblasts; this combination of signaling and structure promotes the formation of new bone.
ii) The matrix is osteoinductive (i.e., actively triggers the formation of bone).
iii) The matrix is osteoconductive (i.e., facilitates the spontaneous formation of bone).
iv) The matrix can be used as a delivery vehicle for agents, such as bone marrow aspirate, platelet-rich plasma or other cellular concentrates; these agents can make the resultant matrix osteogenic (i.e., containing live osteoprogenitor cells that actively promote new bone formation).
v) The matrix can be hydrated, for example with antimicrobial solutions.
vi) The matrix can expand in volume with hydration.
vii) The matrix can be elastic and sponge-like in response to compression, tension, torsion, bending.
viii) The matrix can have different physical characteristics depending on whether the matrix is hydrated or non-hydrated.
ix) The matrix can be capable of mixing with blood, bone marrow aspirate, blood products, and the like.
x) The matrix can be capable of expanding with hydration to fill a void without losing its tensile properties.
xi) The matrix will not dissolve with irrigation or time like other prior materials such as putties, devices and methods.
xii) The matrix will not lose its tensile properties when infiltrated by body fluids.
xiii) The matrix can be capable of conforming to irregular shapes without fracturing or losing basic biologic attributes.
xiv) The matrix can be capable of absorbing blood and fluid and expand with hydration.
xv) The matrix can be capable of tolerating cyclic loading in tension, compression, bending and torsion without early fatigue fracture.
xvi) The matrix can be radiolucent (i.e., permits the penetration and passage of X-rays).

The term “block” or “bone block” as used herein refers to a body of bone material (such as a demineralized cancellous bone matrix) with a side surface that is sized for insertion into a cylindrically-shaped hole of corresponding diameter drilled into bone at a desired operative site as described herein. In an illustrative embodiment, the bone block has a uniform cylindrical or tapered frustoconical side surface. The body also includes a central thru-hole that allows for passage of a fixation member therethrough for guided placement of the block into the drilled hole at the operative site as described herein. The proximal end of the bone block can include articular cartilage. The block is used in a bone grafting surgical procedure that promotes healing of bone and/or cartilage after facture, bone loss, infection, tumor, or other pathologic conditions.

In accordance with the present invention, an instrument kit for bone repair is provided that includes a set of cannulated sizers (1001A, 1001B, . . . 1000N) of different diameters, one or more elongate fixation members 2001 (e.g., Steinman Pin or K-Wire), a set of guides (3001A, 3001B, . . . 3001N) of different diameters (corresponding to the diameters of the sizers (1001A, 1001B, . . . 1001N) and drill bits of the kit), and a set of drill bits (4001A, 4001B, . . . 4001N) of different diameters (corresponding to the diameters of the guides (3001A, 3001B, . . . 3001N) of the kit).

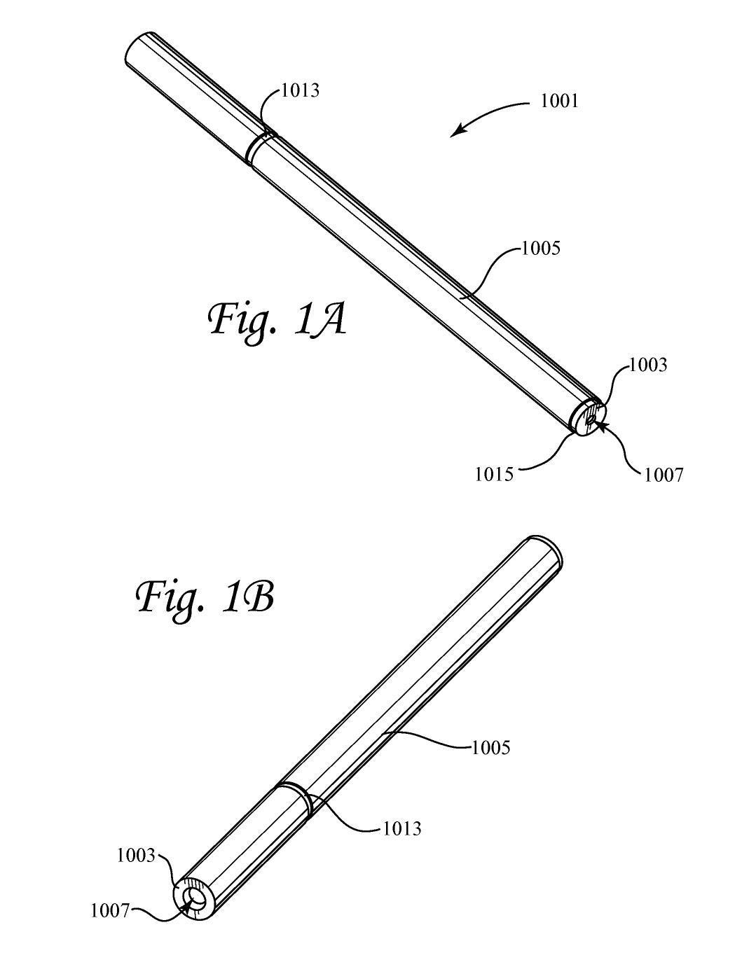

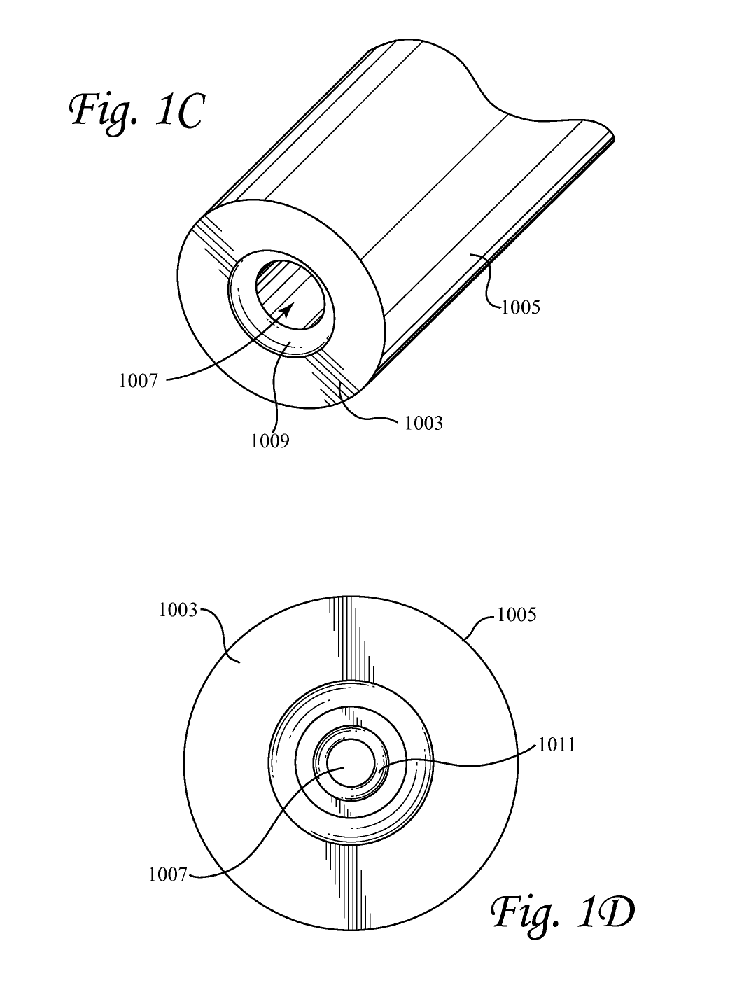

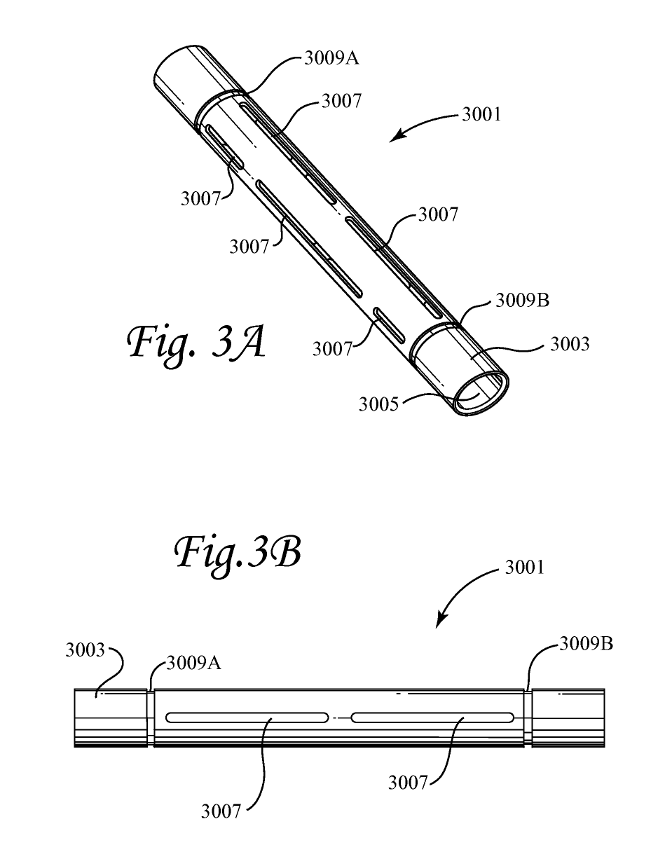

FIGS. 1A-1D illustrate an exemplary sizer 1001 of the kit. As described above, the kit includes a set of sizers (1001A, 1001B, . . . 1001N) of different diameters that are similar in construction to the sizer 1001 of FIGS. 1A-1D. The sizers (1001A, 1001B, . . . 1001N) are used to match to the size of the defective bone at the operative site. In the preferred embodiment, the proper sizer is selected such that it covers the entire bone defect. The proper sizer is also used to push a bone block through the corresponding sized guide 3001 (FIGS. 3A and 3B) along the fixation member 2001 for introduction into a drilled hole into defective bone at the operative site.

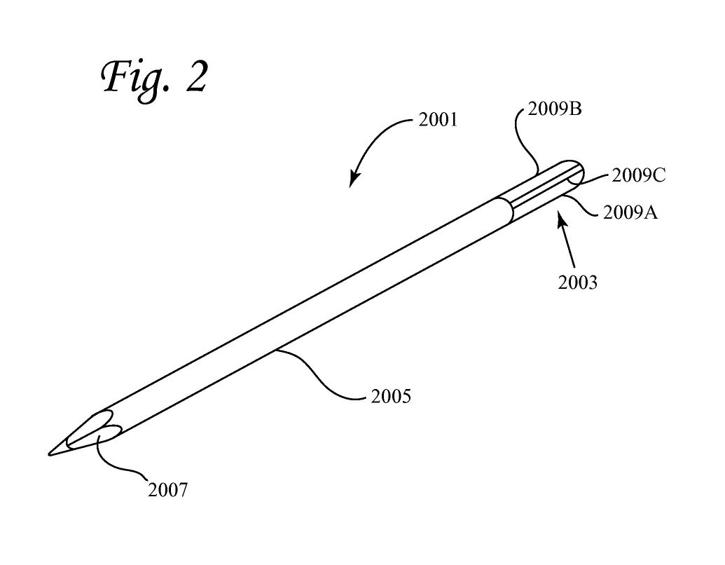

As best shown in FIGS. 1A and 1B, the sizer 1001 includes a rigid body 1003 preferably realized from a hard metal such as titanium, aluminum, or stainless steel. In an illustrative embodiment, the body 1003 is on the order of 190 mm in length; although other lengths can be used. The body 1003 defines a cylindrical outer surface 1005 of predetermined diameter (for example, see the outer diameters of the sizers of the kit as provided in Table I below). The body 1003 also defines a lumen 1005 that extends along the central axis of the sizer 1001 as shown. The lumen 1007 is sized to allow the elongate shaft of the fixation member 2001 (FIG. 2) to pass therethrough. The lumen 1007 preferably has a stepped diameter with a larger diameter along a proximal portion and a smaller diameter along the distal portion. The larger diameter of the proximal portion facilitates entry of the fixation member 2001 into the lumen. The smaller diameter of the distal portion facilitates accuracy in placing the fixation member 2001. The body 1003 can also have a tapered frustoconical design or other suitable design. The size of the sizer 1001 corresponds to the diameter of the distal end of the sizer 1001.

As shown in FIG. 1C, the entrance to the lumen 1007 at the proximal end of the sizer 1001 is chamfered to provide a symmetrical sloping surface 1009 leading to the proximal end of the lumen to facilitate entry of the fixation member 2001 (FIG. 2) into the lumen 1005.

As shown in FIG. 1D, the exit of the lumen at the distal end of the sizer 1001 is recessed and is also chamfered to provide a symmetrical sloping surface 1011 leading away from the distal end of the lumen 1005 to facilitate insertion of the proximal end of the fixation member 2001 later in the procedure as described herein. In this manner, with the proper sizer 1001 covering the defective bone at the operative site, the lumen 1005 of the sizer allows for guided movement of the distal portion of the fixation member 2001 to the defective bone for insertion of the distal end of the fixation member into the central region of the defective bone.

Turning back to FIGS. 1A and 1B, a circumferential groove or band 1013 is preferably defined at a particular position along the length of the sizer 1001. The groove or band 1013 can be painted or otherwise inscribed with a color coded for the respective size (e.g., red for 6 mm, yellow for 8 mm, green for 10 mm, blue for 12 mm) of the sizer 1001. In this manner, the color code of the groove or band 1013 provides a visual indication of the size of the sizer 1001. In the embodiment shown, the groove or band 1013 is defined at approximately 50 mm from the proximal end of the sizer 1001. As will be described in more detail hereinafter, the groove or band 1013 is used to insert a bone block at a desired depth into a drilled hole into defective bone. In the illustrative embodiment, as a bone block is pushed along the fixation member 2001 (FIG. 2) through the corresponding guide 3001 (FIGS. 3A and 3B) by the distal end of the sizer 1001, the groove or band 1013 on the sizer 1001 meets the top end of the guide 3001 (FIGS. 3A and 3B). At this point, the distal end of the sizer 1001 is at a desired depth (for example, 2 mm) below the lower end of the guide 3001 (FIGS. 3A and 3B), thereby “seating” the bone block in the drilled hole, after which it can “rebound” to assume a near-flush orientation with respect to the “top” of the drilled hole.

In one embodiment, the distal end portion 1015 of the sizer 1001 (for example, the distal 2 mm of the sizer) has a slight reduction (e.g., 0.5 mm) in the diameter as is best shown in FIG. 1A. This reduced diameter facilitates entry of the sizer 1001 into the drilled hole in the bone for seating the bone block into the drilled hole, so that the bone block is not damaged by pinching or other interaction between the sizer 1001 (FIGS. 1A and 1B) and the guide 3001 (FIGS. 3A and 3B).

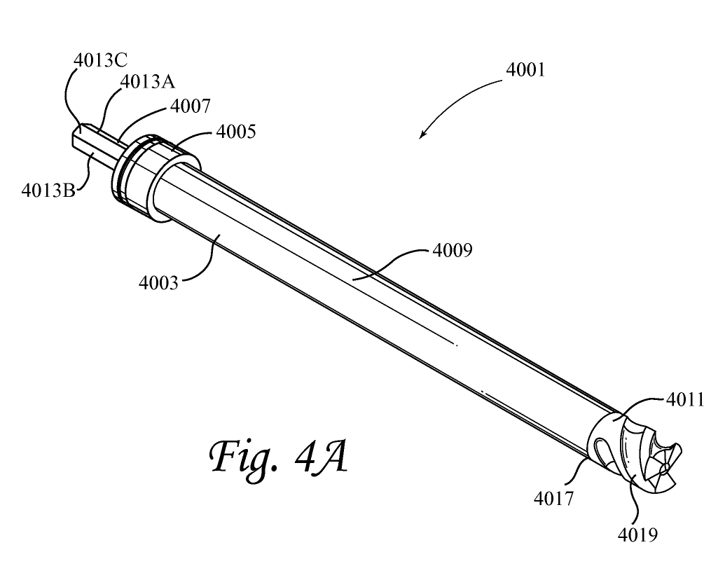

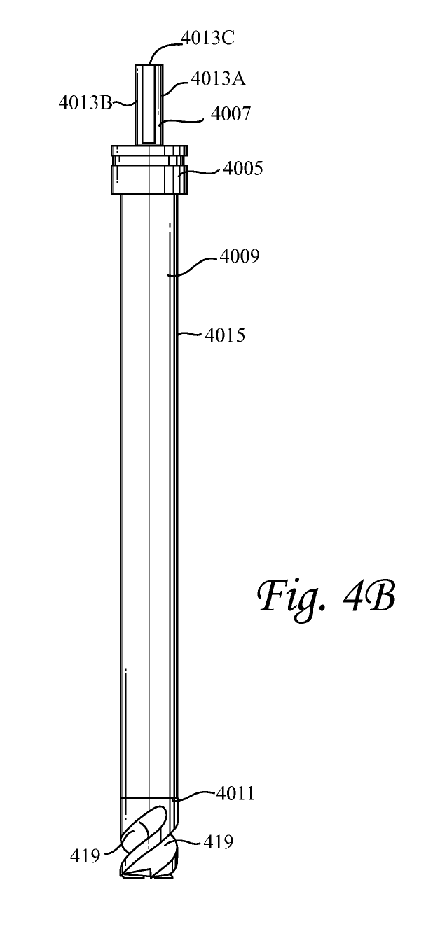

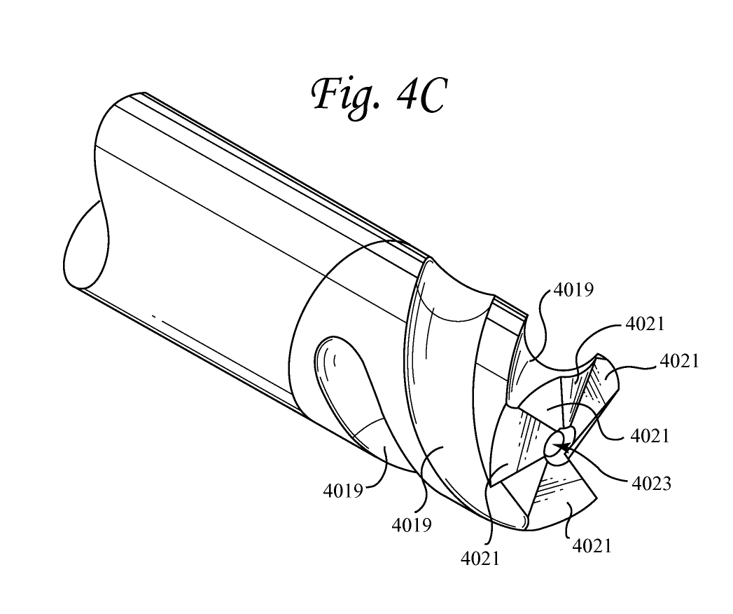

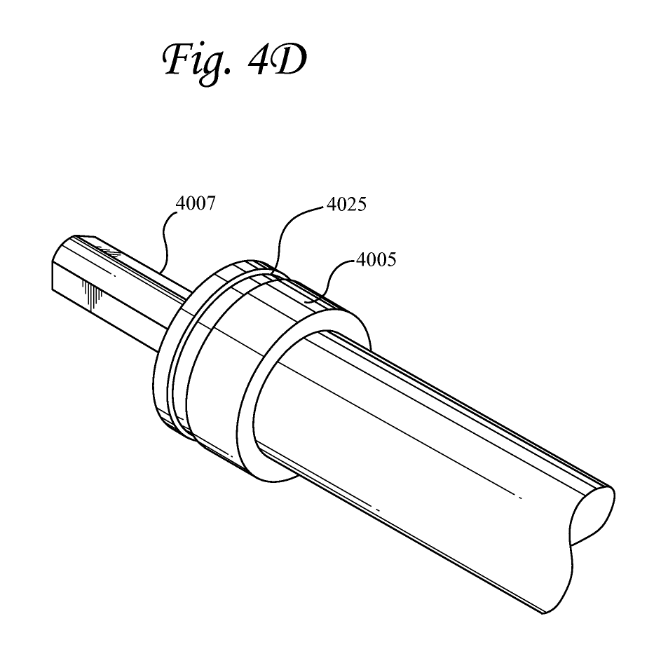

Turning now to FIG. 2, the fixation member 2001 of the kit is preferably realized by a pin (preferably a Kirschner wire) on the order of 200-275 mm in length. The pin 2001 defines a proximal head portion 2003 and an elongate distal portion 2005 terminating in a sharpened insertion end 2005. The proximal head portion 2003 interfaces to an inserter device (not shown) that is used to drive the insertion end 2005 of the pin 2001 into the defective bone as described herein. With the insertion end 2005 inserted into bone, the elongate shaft (head portion 2003 and distal portion 2005) of the pin 2001 is used as a centralizer for guided movement of various parts of the kit, including a drill bit 4001 (FIGS. 4A and 4B) for bone drilling, and a sizer 1001 (FIGS. 1A and 1B) for introduction of the bone block into the hole drilled into the defective bone. Other suitable fixation members such as K wires and Steinman pins can be used as well. The kit can include one or more fixation members as described above.

FIGS. 3A and 3B illustrate an exemplary guide 3001 of the kit. As described above, the kit includes a set of guides of different diameters (corresponding to the diameters of the sizers of the kit). The guides of the kit are similar in construction to the guide 3001 of FIGS. 3A and 3B. The proper guide of the kit is selected that matches the proper sizer of the kit that covers the defective bone. The proper guide is used for guided movement of various parts of the kit, including a drill bit 4001 (FIGS. 4A and 4B) for bone drilling, and a sizer 1001 (FIGS. 1A and 1B) for introduction of the bone block into the hole drilled into the defective bone. It also provides for guided movement of the bone block itself for introduction into the hole drilled into the defective bone.

The guide 1001 includes a thin walled cylindrical body 3003 that is open at both its proximal and distal ends. In an illustrative embodiment, the cylindrical body 3003 is realized from a metal such as titanium, aluminum, or stainless steel and is on the order of 137 mm in length, although other lengths can be used. The thickness of the cylindrical body 3003 is preferably on the order of 1.5 mm, although other thicknesses can be used. The cylindrical body 3003 defines an inner surface 3005 of a predetermined diameter that matches the outer diameter of a corresponding sizer (FIGS. 1A-1D) and drill bit (FIGS. 4A-4D) of the kit such that respective sizer or drill bit can pass through the interior of the body 3003 in contact with or in close proximity to the inner surface 3005 for guided movement of the respective sizer or drill bit therethrough (for example, see the diameters of the guides of the kit as provided in Table I below).

In one embodiment, the cylindrical body 3003 has a number of sets of narrow elongate windows 3007 that extend parallel to the central axis of the body 3007 as shown. The windows 3007 allow for visualization of the interior space of the guide body 3003 during use. This allows for visualization of the bone graft as it moves down the guide body 3003 during insertion. The length and location of the windows 3007 along the cylindrical body 3003 are adapted to balance internal stresses of the guide material and assist in tissue debris control, while providing visibility to assure that the graft is not compressing or losing proper alignment during insertion. In one embodiment, some of the windows 3007 are spaced apart from one another in a direction parallel to the central axis of the cylindrical body 3003, and other windows 3007 are spaced apart from one another about the circumference of the cylindrical body 3003 as best shown in FIG. 3A. For smaller sized guides (e.g., 6 mm and 8 mm guides), three sets of windows 3007 may be offset from one another at 120 degree intervals about the circumference of the respective guide body 3003. For larger sized guides (e.g., 10 mm and 12 mm guides), four sets of windows 3007 may be offset from one another at 90 degree intervals about the circumference of the respective guide body 3003. The edges of the windows 3007 can be chamfered and/or deburred to facilitate progression of the bone graft through the guide body 3003 without impediment.

In the embodiment shown, the body 3003 has one or more circumferential grooves or bands (two shown and labeled 3009A and 3009B, respectively) that are color coded for the respective size (e.g., red for 6 mm, yellow for 8 mm, green for 10 mm, blue for 12 mm) of the guide 3001, although coloring may be applied to one or all surfaces of the instrument. In this manner, the color code of the one or more grooves or bands 1009A, 1009B provide a visual indication of the size of the guide 3001.

FIGS. 4A-4D illustrate an exemplary drill bit 4001 of the kit. As described above, the kit includes a set of drill bits of different diameters (corresponding to the diameters of the guides of the kit). The drill bits of the kit are similar in construction to the drill bit 4001 of FIGS. 4A-4D. The proper drill bit of the kit is selected that matches the proper sizer of the kit that covers the defective bone. The proper drill bit is used for drilling into the defective bone to form a hole that receives a corresponding sized bone block.

The drill bit 4001 is preferably from a two-part design including a rigid mandrel 4003 and a collar 4005. The collar 4005 slides over and is secured to the mandrel 4003 near its proximal end by an interference fit or other suitable fastening means as is well known in the mechanical arts. The mandrel 4003 has a proximal section 4007, an intermediate shaft section 4009 and a distal cutting section 4011. In the preferred embodiment, the total length of the mandrel 4003 is approximately 172 mm (although other lengths can be used), with the proximal section 4007 have a length of approximately 27 mm, the intermediate shaft section 4009 having a length of approximately 128 mm, and the distal cutting section 4011 having a length of approximately 17 mm. Thus, the total length of the intermediate shaft section and distal cutting section is approximately 145 mm, which is greater than the length of the guide (e.g., 137 mm). This ensures that there is clearance between the collar 4005 of the drill bit 4001 and the proximal end of the guide 3001 during the bone boring drilling operation as described herein, resulting in a defect bore depth that correlates to the bone graft height. Alternatively, the drill bit may be constructed from single piece of stock or assembled from discrete parts.

In the embodiment shown in FIGS. 4A and 4B, the proximal section 4007 of the mandrel 4003 defines a plurality of beveled surfaces (some labeled 4013A, 4013B, 4013D) that interface to a driver device (not shown) that is used to rotate the drill bit 4001 for cutting a hole into bone as described herein. A shoulder (not shown) can be provided at the transition between the proximal section 4007 and the intermediate shaft section 4009 of the mandrel 4003. This shoulder can interface to the collar 4005 for fixing the collar 4005 to the mandrel 4003 in the event that the drill bit is produced as an assembly.

The intermediate shaft section 4009 has an outer surface 4015 with a predetermined diameter that matches the diameter of the inner surface 3005 of the corresponding guide (FIGS. 3A and 3B) such that intermediate shaft section 4009 can pass through the interior of the body 3003 in contact with or in close proximity to the inner surface 3005 for guided movement of the respective drill bit 4001 through the guide (for example, see the outer diameters of the drill bits of the kit as provided in Table I below).

The distal cutting section 4011 has an outer surface 4017 with a predetermined diameter that is slightly smaller (preferably on the order of 0.2 mm) than the diameter of the outer surface 4015 of the intermediate shaft section 4009. This reduced diameter minimizes contact between the distal cutting section 4011 and the inner surface 3005 of the guide tube during the bone cutting operation as described herein.

The distal cutting section 4011 also has a set of spiral cutting flutes 4019 that extend from distal surfaces of the cutting section 4011. As best shown in FIG. 4C, these distal surfaces (labeled 4021) are oriented in respective planes that are substantially transverse to the central axis of the drill bit 4001. In an illustrative embodiment, the distal surfaces 4021 are oriented in planes between 70-85 degrees relative to the central axis of the drill bit (more preferably between 75-80 degrees relative to the central axis of the drill bit). These transverse distal surfaces 4021 and the cutting flutes 4019 that extend therefrom produce a substantially flat surface at the bottom of the hole bored by cutting flutes 4019 of the distal cutting section 4011. The shape of the hole bored by the distal cutting section 4011 is intended to mate with the shape of a bone graft of corresponding diameter.

As best shown in FIG. 4C, the mandrel 4003 also has a lumen 4023 that extends along the central axis of the mandrel 4003 over its entire length and is sized to allow the elongate shaft of the fixation member 2001 (FIG. 2) to pass therethrough. The lumen 4023 preferably has a stepped diameter with a larger diameter along a proximal portion and a smaller diameter along a distal portion. The larger diameter of the proximal portion facilitates entry of the fixation member 2001 into the lumen 4023. The smaller diameter of the distal portion facilitates accurate positioning of the drill bit during drilling.

The distance between the distal surface of the drill bit collar and the distal end of the drill bit are such that the exposed length of the drill bit when the collar is in contact with the appropriate guide are the desired depth of the drilled defect bore. Hence, advancement of the drill is impeded when the collar contacts the guide, resulting in a final bore depth that correlates to the bone graft height.

In the embodiment shown, the collar 4005 of the drill bit 4001 has a circumferential groove or band 4025 as best shown in FIG. 4D. The groove or band 4025 is color coded for the respective size (e.g., red for 6 mm, yellow for 8 mm, green for 10 mm, blue for 12 mm) of the drill bit 4001. In this manner, the color code of the groove or band 4025 provides a visual indication of the size of the drill bit 4001.

Preferably, matching colors are imparted to the grooves or bands for the matching parts (sizer, drill bit, guide) of the kit for a given size, and these colors are distinct for the different sized parts of the kit as is evident from the exemplary kit embodiment of Table I below. Additionally or alternatively, exerior surfaces of one or more instrument may be marked with the appropriate size identification color to match the other similarly sized instruments in the kit. The matching sized parts are also preferably etched with alphanumeric characters that indicate the appropriate size of the parts.

TABLE I

| | | Drill Bit OD | | |
|---|---|---|---|---|
| | | (Shaft/Cutting | | |
| Tool Size | Sizer OD | Section) | Guide ID | Band Color |
| 6 mm | 6 mm | 5.95 mm/5.73 mm | 6.5 mm | Red |
| 8 mm | 8 mm | 7.95 mm/7.73 mm | 8.5 mm | Yellow |
| 10 mm | 10 mm | 9.95 mm/9.73 mm | 10.5 mm | Green |
| 12 mm | 12 mm | 11.95 mm/11.73 mm | 12.5 mm | Blue |

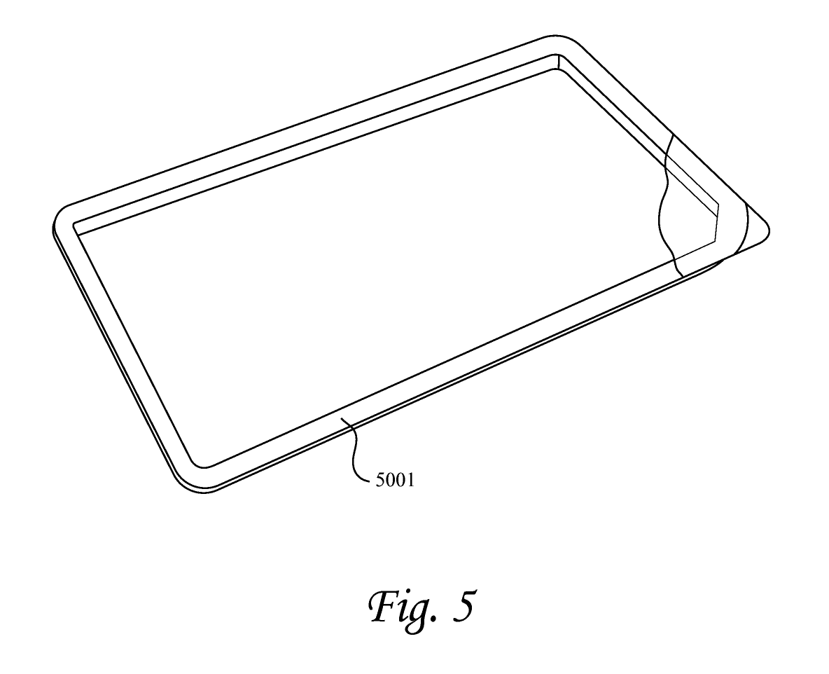

In one embodiment, the instruments of a kit of the present invention, including a set of cannulated sizers (1001A, 1001B, . . . 1001N) of different diameters, one or more elongate fixation members 2001, a set of guides (3001A, 3001B, . . . 3001N) of different diameters (corresponding to the diameters of the sizers and drill bits of the kit), and a set of drill bits (4001A, 4001B, . . . 4001N) of different diameters (corresponding to the diameters of the guides of the kit) as described herein, are housed in one or more enclosures, such as an instrument tray 5001 as shown in FIG. 5, that provides the surgeon easy access to the instruments as needed. The enclosure can be realized from suitable material (such as stainless steel) that allows for repeated cleaning and sterilization of the instruments of the kit by desired cleaning methods (e.g., water and enzyme washing, etc.) and desired sterilization methods (e.g., steam, autoclave, dry heat, chemical, etc.).

The kit of the present invention as described herein is used in a bone grafting surgical procedure to promote healing in defective bone, e.g., in the subchondral region of articulating joints. The kit can also be used to establish and maintain a port of entry for arthroscopic instruments or for an open procedure. The kit is also used to introduce a bone block to the operative site during the surgical procedure.

According to one aspect of the invention, a surgical method is provided utilizing the previously described surgical kit. The surgical method begins by making an incision to gain access to the operative site. The cannulated sizers (1001A, 1001B, . . . 1001N) of the kit are used to identify the proper sizer that covers the defective bone at the operative site as measured by diameter. This is accomplished by inserting one or more of the sizers through the incision such that the distal end of the respective sizer is positioned near or adjacent the defective bone, and making a visual determination whether the respective sizer covers the bone defect. Increasingly larger sizers can be positioned near or adjacent the defective bone as necessary. The proper sizer covers the entire defect.

With the proper sizer 1001 centered over the bone defect, the fixation member 2001 is then introduced through the lumen 1007 of the sizer 1001, and the fixation member 2001 can be secured in place into the central region of the bone defect with a suitable introducer (e.g., surgical drill or the like). When secured in place, the sharp distal end of the fixation member 2001 is anchored to the bone such that it does not move. This ensures proper orientation and placement of the fixation member 2001.

Next, the proper guide 3001 of the kit that is matched to the size of the proper sizer 1001 is selected and placed over the proper sizer 1001 (with the fixation member 2001 extending through the lumen of the proper sizer 1001). For example, an 8 mm guide is selected in the event that the 8 mm sizer is used as the proper sizer for the procedure. As described above, the instruments are color coded and etched with the size for ease of use.

Next, the proper sizer 1001 is removed leaving the fixation member 2001 and the guide 3001 in place for the remainder of the procedure.

Next, the proper drill bit 4001 of the kit that is matched to the size of the proper sizer 1001 is selected and loaded onto a surgical power drill. For example, an 8 mm drill bit is selected in the event that the 8 mm sizer is used as the proper sizer for the procedure. As described above, the instruments are color coded and etched with the size for ease of use. The proper drill bit 4001 is placed over the proximal end of the fixation member 2001 (which is centrally fixed to the defective bone at its distal end) and moved down into the proper guide 3001. The power drill is then activated to rotate the proper drill bit 4001 and the rotating drill bit 4001 is used to drill into the defective bone that surrounds the distal end of the fixation member 2001 until the stop mechanism of the proper drill bit 4001 contacts the proper guide 3001. This stop mechanism assures that the correct drill depth is achieved. Debris can be removed from drill bit 4001 and guide 3001 as necessary. After completing the drilled hole, the proper drill bit is slid proximally out of the proper guide 3001 and off the proximal end of the fixation member 2001.

Next, a proper bone block is selected that is sized to match the drilled hole into the defective bone. For example, an 8 mm bone block is selected for use with an 8 mm sized drilled hole. The proper bone block can be provided in a sterile package and hydrated for use as appropriate. The proper bone block can be loaded with agents, such as bone marrow aspirate, platelet-rich plasma or other cellular concentrates. These agents can make the bone block osteogenic (i.e., containing live osteoprogenitor cells that actively promote new bone formation). A sterile field is prepared around the operative site as appropriate. The proper bone block (preferably hydrated for introduction into the defective bone) is placed over the proximal end of the fixation member 2001 and moved down toward and preferably into the proper guide 3001. The thru-hole of the proper bone block receives the elongate shaft of the fixation member 2001 and allows the proper bone block to slide along the elongate shaft of the fixation member 2001.

Next, with the proper bone block mounted on the fixation member 2001, the proper sizer 1001 is placed over the proximal end of the fixation member 2001 and moved down toward the proper guide 3001. The proper sizer 1001 is advanced distally down into the proper guide 3001 such that it pushes the proper bone block down the proper guide 3001 and into the drilled hole. Preferably, the bone block is viewed through the windows of the proper guide 3001 during advancement to assure proper alignment of the spongy block; i.e., that it enters the hole properly. The color coded groove or band on the proper sizer 1001 can be used to ensure that the proper bone block is seated at the correct depth as described herein. More specifically, the proper bone block is seated to the proper depth when the color coded groove or band on the proper sizer 1001 meets the top of the proper guide 3001.

Finally, the fixation member 2001 is removed from the operative site, followed by removal of the proper sizer 1001, and then followed by removal of the proper guide 3001.

There have been described and illustrated herein embodiments of a surgical instrument kit and an associated bone grafting surgical method. Advantageously, the surgical instrument kit and bone graft surgical method of the present invention as described herein allows the surgeon to effectively and efficiently identify the size of the bone defect, remove defective bone, size the bone block for the defect and accurately place the bone block within the defect. It is particularly suited for bone grafting surgical procedures that promote healing in defective bone and/or cartilage in the subchondral region of articulating joints, but it is not limited to this particular type of procedure and can be used in other bone grafting surgical procedures. It is also particularly suited for introducing a bone block into defective bone and/or cartilage, but it is not limited to this particular type of bone graft and can be used to introduce other types of bone grafts, such as autograft bone, allograft bone, synethic bone material, and xenogeneic bone material. The bone graft is preferably cannulated with a central thru-hole that allows for passage of a fixation member therethrough for guided placement of the bone graft into the drilled hole at the operative site as described herein. While particular embodiments of the invention have been described, it is not intended that the invention be limited thereto, as it is intended that the invention be as broad in scope as the art will allow and that the specification be read likewise. Thus, while particular dimensions and kit configurations have been disclosed, it will be appreciated that other dimensions and kit configurations can be used as well. In addition, while particular types of materials have been disclosed, it will be understood that other suitable materials can be used. Furthermore, while particular surgical methodologies have been disclosed in reference to using the kit for bone repair, it will be appreciated that the surgical instruments of the kit can be used for other surgical methods for bone repair as well. It will therefore be appreciated by those skilled in the art that yet other modifications could be made to the provided invention without deviating from its spirit and scope as claimed.

What is claimed is:

1. A kit of surgical instruments for bone repair comprising:
a set of sizer instruments defined by bodies of varying diameters;
a set of drill bits for boring into bone, the drill bits of varying diameters that correspond to the sizer instruments of the kit; and
a set of guide instruments defined by thin-walled cylindrical bodies of varying diameters that correspond to the diameters of the sizer instruments and drill bits of the kit, wherein each cylindrical body includes an open proximal end and an open distal end and has an inner cylindrical surface sized to provide for translating movement of a corresponding sizer instrument and drill bit instrument therethrough;
wherein the sizer instruments, the drill bits and the guide instruments of the kit each have a feature that is color coded to provide a visual indication of corresponding sized instruments of the kit.

2. A surgical kit according to claim 1, wherein:
a respective color is painted or otherwise inscribed on the feature of corresponding sized sizer instruments, drill bits, and guide instruments of the kit to provide a visual indication of corresponding sized instruments of the kit.

3. A surgical kit according to claim 1, wherein:
the feature is defined about the circumerference of an outer surface of a respective instrument of the kit.

4. A surgical kit according to claim 3, wherein:
the feature comprises a band or groove that extends about the circumference of the outer surface of the respective instrument of the kit.

5. A surgical kit according to claim 1, further comprising:
alphanumeric characters that convey size being marked on the corresponding sized sizer instruments, drill bits, and guide instruments of the kit to provide a visual indication of corresponding sized instruments of the kit.

6. A surgical kit according to claim 1, wherein:
the sizer instruments and the drill bits of the kit each have a central lumen that extends along their respective entire length.

7. A surgical kit according to claim 6, wherein:
the central lumen of the respective instrument allows for passage of the respective instrument over an elongate fixation member, the fixation member having a distal end that is adapted to be fixed into bone.

8. A surgical kit according to claim 7, further comprising:
at least one fixation member that is received within the central lumens of the sizers and drill bits of the kit.

9. A surgical kit according to claim 1, wherein:
each given drill bit of the kit has a plurality of distal surfaces that are oriented in respective planes that are substantially transverse to the central axis of the given drill bit such that given drill bit cuts a hole into bone wherein the hole has a substantially flat bottom surface.

10. A surgical kit according to claim 1, wherein:
each given guide instrument of the kit includes at least one window in the cylindrical body of the given guide instrument, wherein the window provides for visualization into the interior space of the given guide instrument during use.

11. A surgical kit according to claim 10, wherein:
the at least one window is narrow in shape and extends parallel to the central axis of the cylindrical body of the given guide instrument.

12. A surgical kit according to claim 11, wherein:
the at least one window comprises a plurality of windows that are narrow in shape and extend parallel to the central axis of the cylindrical body of the given guide instrument.

13. A surgical kit according to claim 12, wherein:
some of the windows are spaced apart from one another in a direction parallel to the central axis of the cylindical body of the given guide instrument, and other windows are spaced apart from one another about the circumference of the cylindrical body of the given guide instrument.

14. A surgical kit according to claim 12, wherein:
the edges of the plurality of windows are chamfered and/or deburred.

15. A surgical kit according to claim 1, further comprising:
an enclosure for housing the instruments of the kit.

16. A kit of surgical instruments for bone repair comprising:
a set of sizer instruments defined by cylindrically shaped or tapered/conical bodies of varying diameters;
a set of drill bits for boring into bone, the drill bits of varying diameters that correspond to the sizer instruments of the kit; and
a set of guide instruments defined by thin walled cylindrical bodies of varying diameters that correspond to the diameters of the sizer instruments and drill bits of the kit, wherein each cylindrical body includes an open proximal end and an open distal end and has an inner cylindrical surface sized to provide for translating movement of a corresponding sizer instrument and drill bit instrument therethrough;
wherein each given drill bit has a plurality of distal surfaces that are oriented in respective planes that are substantially transverse to the central axis of the given drill bit such that given drill bit cuts a hole into bone wherein the hole has a substantially flat bottom surface; and
wherein the sizer instruments and the drill bits of the kit each have a central lumen that extends along their respective entire length.

17. A surgical kit according to claim 16, wherein:
the central lumen of the respective instrument allows for passage of the respective instrument over an elongate fixation member, the fixation member having a distal end that is adapted to be fixed into bone.

18. A surgical kit according to claim 17, further comprising:
at least one fixation member that is received within the central lumens of the sizers and drill bits of the kit.

19. A surgical kit according to claim 16, further comprising:
an enclosure for housing the instruments of the kit.

20. A kit of surgical instruments for bone repair comprising:
a set of sizer instruments defined by cylindrically shaped bodies of varying diameters;
a set of drill bits for boring into bone, the drill bits of varying diameters that correspond to the sizer instruments of the kit; and
a set of guide instruments defined by thin walled cylindrical bodies of varying diameters that correspond to the diameters of the sizer instruments and drill bits of the kit, wherein each cylindrical body includes an open proximal end and an open distal end and has an inner cylindrical surface sized to provide for translating movement of a corresponding sizer instrument and drill bit instrument therethrough;
wherein each given guide instrument includes at least one window in the cylindrical body of the given guide instrument, wherein the window provides for visualization into the interior space of the given guide instrument during use.

21. A surgical kit according to claim 20, wherein:
the at least one window is narrow in shape and extends parallel to the central axis of the cylindrical body of the given guide instrument.

22. A surgical kit according to claim 21, wherein:
the at least one window comprises a plurality of windows that are narrow in shape and extend parallel to the central axis of the cylindrical body of the given guide instrument.

23. A surgical kit according to claim 22, wherein:
some of the windows are spaced apart from one another in a direction parallel to the central axis of the cylindical body of the given guide instrument, and other windows are spaced apart from one another about the circumference of the cylindrical body of the given guide instrument.

24. A surgical kit according to claim 22, wherein:
the edges of the plurality of windows are chamfered and/or deburred.

25. A surgical kit according to claim 20, further comprising:
an enclosure for housing the instruments of the kit.

26. A surgical method of repair of a bone defect comprising:
providing a surgical kit comprising a set of sizer instruments, a set of drill bits for boring into bone, and a set of guide members, wherein the sizer instruments of the kit are defined by cylindrically shaped bodies of varying diameters, wherein the drill bits of the kit have varying diameters that correspond to the sizer instruments of the kit, and wherein the guide instruments of the kit are defined by thin walled cylindrical bodies of varying diameters that correspond to the diameters of the sizer instruments and drill bits of the kit, wherein each cylindrical body includes an open proximal end and an open distal end and has an inner cylindrical surface sized to provide for translating movement of a corresponding sizer instrument and drill bit instrument therethrough;
using the sizer instruments of the kit to identify a proper sizer instrument that covers the bone defect;
selecting a proper guide instrument and a proper drill bit of the kit that matches the proper sizer instrument; and
using the proper guide instrument to guide movement of the proper drill bit during coring of the bone defect in order to drill a hole in the bone defect.

27. A surgical method according to claim 26, further comprising:
selecting a bone graft sized for the hole drilled in the bone defect, and using the proper guide instrument in order to guide introduction of the bone graft into the hole drilled in the bone defect.

28. A surgical method according to claim 27, further comprising:
pushing the bone graft with the proper sizer through the proper guide instrument in order to introduce the bone graft into the hole drilled in the bone defect.

29. A surgical method according to claim 26, wherein:
the sizer instruments and the drill bits of the kit each have a central lumen that extends along their respective entire length, wherein the central lumen of the respective instrument allows for passage of the respective instrument over an elongate fixation member.

30. A surgical method according to claim 29, further comprising:
inserting a fixation member through the lumen of the proper sizer instrument and securing the distal end of the fixation member in a central region of the bone defect.

31. A surgical method according to claim 30, wherein:
after the distal end of the fixation member is secured in the central region of the bone defect, the proper drill bit is moved over the fixation member during coring of the bone defect in order to drill a hole in the bone defect.

32. A surgical method according to claim 30, wherein:
after the distal end of the fixation member is secured in the central region of the bone defect, the proper sizer is moved over the fixation member in order to push the bone graft with the proper sizer through the proper guide instrument for introducing the bone graft into the hole drilled in the bone defect.

33. A surgical method according to claim 29, wherein:
the surgical kit further includes at least one fixation member that is received within the central lumens of the sizers and drill bits of the kit.

34. A surgical method according to claim 26, wherein:
the defective bone is in the subchondral region of an articulating joint.

35. A surgical method according to claim 27, wherein:
the bone graft is a body of bone material with a side surface sized for insertion into the hole drilled into the bone defect.

36. A surgical method according to claim 35, wherein:
body of bone material of the bone graft defines a central thru-hole that allows for passage of a fixation member therethrough for guided placement of the body of bone material into the hole drilled into the bone defect.

37. A surgical method according to claim 35, wherein:
the bone material of the bone graft comprises demineralized cancellous bone matrix.

38. A surgical method according to claim 27, wherein:
the bone graft comprises demineralized cancellous bone matrix.

39. A surgical method according to claim 27, wherein:
the bone graft comprises material selected from the group consisting of: autograft bone, allograft bone, demineralized cancellous bone matrix, a synthetic bone product, and a xenogeneic bone product.

40. A surgical method according to claim 28, wherein said proper guide instrument includes a thin walled cylindrical body defining at least one visualization window, said method further comprising:
viewing the bone graft through the visualization window during said pushing to assure proper alignment of the bone graft.

41. A surgical instrument for bone repair comprising:
at least one drill bit for boring into bone, the drill bit having a plurality of distal surfaces that are oriented in respective planes that are substantially transverse to the central axis of the drill bit such that drill bit cuts a hole into bone wherein the hole has a substantially flat bottom surface, and a central lumen that extends along the entire length of the drill bit, wherein the central lumen of the drill bit allows for passage of the drill bit over an elongate fixation member.
